(19) United States
(12) Plant Patent Application Publication
(10) Pub. No.: US 2003/0145363 P1
(43) Pub. Date: Jul. 31, 2003

(54) Calibrachoa plant named 'Kakegawa S49'

(76) Inventor: Masao Bessho, Kakegawa City (JP)

Correspondence Address: JONDLE & ASSOCIATES P.C., 9085 EAST MINERAL CIRCLE, SUITE 200, CENTENNIAL, CO, 80112, US

(21) Appl. No.: 10/058,848

(22) Filed: Jan. 30, 2002

(51) Int. Cl. A01H 5/00

(52) U.S. Cl. PLT/263

(57) ABSTRACT

‘Kakegawa S49’ is a new variety of Calibrachoa. This new variety has light purple violet colored flowers and dark green foliage.

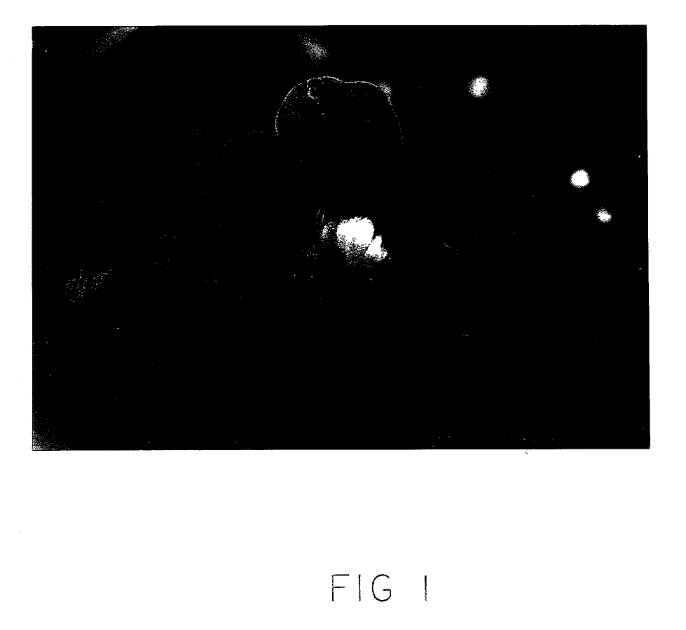

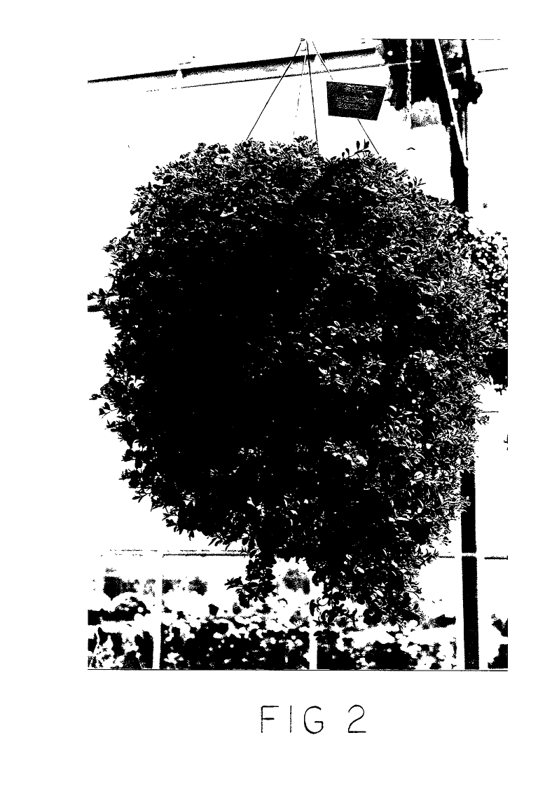

### BACKGROUND OF THE INVENTION

‘Kakegawa S49’ originated from a hybridization made in spring 1996 in Kakegawa, Japan. The female parent was the commercial variety ‘Liricashower Rose’ (U.S. Plant Pat. No. 9,884). The male parent was the commercial variety ‘Million Bells Trailing White’ (U.S. Plant Pat. No. 10,278). Seed from this cross was sown in spring 1997 and four F₁ plants were selected. The four selections were intercrossed and the resulting seed bulked together to produce F₂ seed. The F₂ seed was sown in summer 1997 and two plants were selected for having sky blue flower color. The selections were intercrossed and the resulting seed bulked together to produce F₃ seed. In spring 1998 the F₃ seed was sown and one plant, designated as line K9-119, was selected. This line was vegetatively propagated in Salinas, Calif. in summer 1998 and again in spring 1999. In these two vegetative generations the line was evaluated and determined that the traits are firmly fixed and stable. No inherent variation or off-types have been identified.

### DESCRIPTION OF THE GENUS CALIBRACHOA LLAVE & LEX

The genus Petunia was originally established in 1803 by A. L. Jussieu, who described both P. parviflora and P. nyctaginifloa as type species. Using a non-horticultural system that selected the first mentioned species as the type species (lectotype), N. L. Britton and H. A. Brown declared P. parviflora as the type species for Petunia in 1913.

During the 1980's and 1990, H. J. Wijsman published a series of articles regarding the ancestry of P. hybrida, the Garden Petunia, and the inter-relationship of several species classified as Petunia. These studies discovered that P. hybrida and its ancestrial species, P. nyctaginiflora (=P. axillaris) and P. violacea (=P. integrifolia), possessed 14 pairs of chromosomes while several other species, including P. parviflora, possessed 18 pairs of chromosomes. Since P. parviflora was the lectotype species for the Petunia genus, Wijsman and J. H. de Jong proposed transferring the 14 chromosome species to the genus Stimoryne. Horticulturists opposed reclassifying the Garden Petunia and in 1986, Wijsman proposed the alternative of making P. nyctaginiflora the lectotype species for Petunia and transferring the 18 chromosome species to another genus. The I. N. G. Committee adopted this proposal. By 1990 Wijsman had transferred several species, including P. parviflora (=C. parviflora) to Calibrachoa, originally established by Liave and Lexarza in 1825. Calibrachoa parviflora (=C. mexicana Llave & Lexarza) is now the type species for the genus Calibrachoa.

Classification of the current Petunia and Calibrachoa species is still in progress. New species are also being identified. Consequently a proper description has not been written for the Calibrachoa genus. Calibrachoa can, however, be distinguished from Petunia based on the higher chromosome number, chromosome morphology, plant branching habit and type of flower bud aestivation. Whereas Petunia species bear a flower peduncle and one new stem from a node, Calibrachoa bear a flower peduncle and three stems. Petunia species have a cochlear corolla bud, a single outermost petal covers the other four, radially folded and terminally contorted petals. Calibrachoa flower buds are flat with all five petals linearly folded and the two lower petals forming a cover around the three other petals and fused together.

### ENVIRONMENTAL CONDITIONS FOR PLANT GROWTH

Vegetative cuttings were propagated in five to six weeks. Cuttings were stuck into plastic cell trays having 98 cells, and containing a peat moss-based growing medium. The base of the cuttings were dipped for 1 to 2 second sin a 1:9 solution of Dip 'N Grow (1 part solution to 9 parts water) root inducing solution immediately prior to sticking into the cell trays. The cuttings were misted with water from overhead for 10 seconds every 30 minutes until sufficient roots were formed.

Rooted cuttings were transplanted and grown in 20 cm diameter plastic pots in a glass greenhouse located in Salinas, Calif. Pots contained a peat moss-based growing medium. Soluble fertilizer containing 20% nitrogen, 10% phosphorus and 20% potassium was applied once a day or every other day by overhead irrigation. Pots were top-dressed with a dry, slow release fertilizer containing 20% nitrogen, 10% phosphorus and 18% potassium. The typical average air temperature was 24C.

### DETAILED DESCRIPTION OF THE NEW PLANT

The following traits and characteristics describe the new variety.

Classification:

Family.—Solanaceae.

Species.—Calibrachoa spp.

Common names.—Petunia.

Parentage:

Female parent.—Liricashower Rose (U.S. Plant Pat. No. 9,884).

Male parent.—Million Bells Trailing White (U.S. Plant Pat. No. 10,278).

Growth:

Habit.—decumbent, mounding in center.

Height.—23.0 cm.

Spread.—115.0 cm when grown in a 41 cm hanging basket or pot, and using five 20 cm potted plants in one hanging basket.

Life cycle.—annual.

Time to produce a rooted cutting.—3 weeks.

Time to bloom from propagation.—10 weeks.

Form.—branching, dense.

Stems:

Stem color.—R.H.S. 144B (yellow-green).

Anthocyanin color.—R.H.S. N77A (purple).

Pubescence.—slightly.

Pubescence color.—R.H.S. 155D (white).

Pubescence shape.—pointed.

Stem description.—round, ancipital.

Stem diameter.—2.0 mm.

Internode length.—1.5 to 2.5 cm.

Leaves:

Leaf tip.—mucronate.

Leaf arrangement.—alternate.

Leaf base.—decurrent.

Leaf color.—upper surface: R.H.S. 137A (green); lower surface: R.H.S. 138B (green).

Leaf frangrance.—absent.

Leaf margin.—entire.

Leaf surface.—slightly.

Petiole color.—R.H.S. 138C (green).

Petiole length.—2.0 to 3.0 mm.

Leaf length.—3.0 to 3.5 cm.

Leaf shape.—spatulate.

Leaf width.—1.0 cm at full expansion.

Flowers:

Calyx.—5 sepals; 1.0×3.0 cm (l×w); free.

Corolla.—5 petals, fused.

Flower diameter.—3.0 cm.

Bud color.—R.H.S. N144D (yellow-green).

Bud shape.—ovate.

Bud surface.—pubescent.

Duration of flower life.—5 days.

Flowering habit.—indeterminate.

Ovary.—superior.

Placenta arrangement.—central.

Peduncle.—2.5 cm length; 1.0 mm diameter; slightly smooth, short pubescence.

Inflorescence type.—solitary.

Stamens.—5 stamens, three short and two long, free.

Stamens color.—R.H.S. 144D (yellow-green).

Stigma color.—R.H.S. 134A (green).

Style color.—R.H.S. 144D (yellow-green)

Petal size.—2.5×1.5 cm (l×w).

Petal color.—upper surface: R.H.S. N82B (purple-violet); lower surface: R.H.S. 84D (violet); corolla throat: R.H.S. 155D (white) with R.H.S. N77A (purple) veins; corolla tube: inner surface: R.H.S. 4C (yellow); outer surface: R.H.S. 4C (yellow).

Petal pubescence.—glabrous.

Pollen color.—R.H.S. 1A (green-yellow).

Produces seed.—yes.

### COMPARISON WITH MOST SIMILAR VARIETY

‘Kakegawa S49’ is most similar to the variety ‘Colorburst Violet’ (U.S. Plant Pat. No. 12,086). ‘Kakegawa S49’ differs from ‘Colorburst Violet’ in that it has a lighter purple-violet flower color, a pale yellow corolla throat and a more prostrate, trailing plant habit.

What is claimed is:

1. A new and distinct cultivar of Calibrachoa as shown and described herein.
